(19) United States
(12) Patent Application Publication
(10) Pub. No.: US 2023/0372381 A1
(43) Pub. Date: Nov. 23, 2023

(54) COMPOSITIONS AND METHODS UTILIZING PROTEIN INHIBITORS FOR SYNERGISTIC INHIBITION AND TREATMENT OF SARS-CoV-2

(71) Applicant: Rensselaer Polytechnic Institute, Troy, NY (US)

(72) Inventors: Gaetano Thomas MONTELIONE, Manchester Center, VT (US); Khushboo BAFNA, Troy, NY (US); Robert M. KRUG, Gwynedd, PA (US); Aldolfo GARCIA-SASTRE, Troy, NY (US); Kris WHITE, Lynbrook, NY (US)

(73) Assignee: Rensselaer Polytechnic Institute, Troy, NY (US)

(21) Appl. No.: 18/030,366

(22) PCT Filed: Oct. 5, 2021
(86) PCT No.: PCT/US2021/053538

§ 371 Date: Apr. 5, 2023

Related U.S. Application Data
(60) Provisional application No. 63/087,524, filed on Oct. 5, 2020.
(60) Provisional application No. 63/087,766, filed on Oct. 5, 2020.
(60) Provisional application No. 63/124,578, filed on Dec. 11, 2020.
(60) Provisional application No. 63/175,977, filed on Apr. 16, 2021.
(60) Provisional application No. 63/176,445, filed on Apr. 19, 2021.

(51) Int. Cl. A61K 31/7068; A61K 31/675; A61K 31/4965; A61K 31/407; A61K 31/4995; A61K 31/497; A61P 31/14

(52) U.S. Cl. CPC A61K 31/7068; A61K 31/675; A61K 31/4965; A61K 31/407; A61K 31/4995; A61K 31/497; A61P 31/14

(57) ABSTRACT

Compositions discussed herein are useful for treating a patient infected with a coronavirus, notably SARS-CoV-2 or COVID-19. The compositions to be administered to an infected patient utilize inhibitors of SARS-CoV-2 papain-like protease (PL^{pro}). Several of these inhibitors include available hepatitis C virus (HCV) drugs that are already FDA-approved. These PL^{pro} inhibitors have been found to be strongly synergistic with inhibitors of SARS-CoV-2 polymerases, such as RNA-dependent RNA polymerase (RdRp). One such polymerase inhibitor, remdesivir, is already in use as an anti-viral treatment of patients with COVID-19. The PL^{pro} inhibitors, including repurposed HCV drugs such as grazoprevir, vaniprevir, and paritaprevir, increase remdesivir's ability to suppress coronavirus replication antiviral activity as much as 10-fold. These compositions can provide more effective patient therapies with less toxicity and side effects compared with the current standard of care, as well as enable outpatient treatment for COVID-19 with orally ingestible polymerase inhibitors such as molnupiravir (MK-4482/EIDD-2801).

### CROSS REFERENCE TO RELATED APPLICATION(S)

This application is a national stage filing of International Patent Application No. PCT/US2021/053538, filed Oct. 5, 2021, which claims the benefit of U.S. Provisional Application Nos. 63/087,524, filed Oct. 5, 2020; 63/087,766, filed Oct. 5, 2020; 63/124,578, filed Dec. 11, 2020; 63/175,977, filed Apr. 16, 2021; and 63/176,445, filed Apr. 19, 2021, which are incorporated by reference as if disclosed herein in their entireties.

### STATEMENT REGARDING FEDERALLY SPONSORED RESEARCH OR DEVELOPMENT

This invention was made with government support under grant no. GM120574 awarded by the National Institutes of Health. The government has certain rights in the invention.

### BACKGROUND

Coronaviruses (CoVs) cause human respiratory diseases. While several human coronaviruses cause relatively mild respiratory infections, three coronaviruses cause severe respiratory diseases in humans: Severe Acute Respiratory Syndrome virus (SARS), Middle East Respiratory Syndrome virus (MERS), and Corona Virus Infectious Disease 2019 virus (SARS-CoV-2 or COVID-19).

Coronaviruses, including SARS-CoV-2, are enveloped viruses. Their genome is comprised of a single, large (27-34 kilobase) positive-sense single-stranded RNA, which is directly translated by host cells. The SARS-CoV-2 genome encodes 4 structural proteins, 16 non-structural proteins (NSPs) which carry out crucial intracellular functions, and 9 accessory proteins. Many of these proteins, and their host binding partners, are potential targets for development of antivirals for SARS-CoV-2. For example, the repurposed drug remdesivir, which inhibits the viral RNA-dependent RNA polymerase, is the current FDA-approved antiviral standard of care for COVID-19.

Translation of the viral genomic RNA results in the biosynthesis of two polyproteins that are processed into the 16 separate NSPs by two virus-encoded cysteine proteases, the papain-like protease (PL^{pro}) and a 3C-like protease (3CL^{pro}). The latter is also referred to as the main protease (M^{pro}). M^{pro} and PL^{pro} are essential for the virus life cycle and hence are attractive targets for antiviral development. These two viral proteases are used to produce functional viral RNA polymerases. M^{pro} cleavages are predicted to generate several NSPs, including the three subunits NSP7, NSP8, and NSP12 that constitute the viral RNA polymerase complex, as well as integral membrane proteins NSP4 and NSP6. PL^{pro} cleavages generate other NSPs, including NSP3. The NSP3-NSP4-NSP6 complex is a key component of the replication organelles, also known as double membrane vesicles (DMVs), that contribute to the function of the viral polymerase in infected cells.

The COVID-19 pandemic has caused more than four million deaths worldwide and crippled the global economy. Effective control of the SARS-CoV-2 coronavirus that causes COVID-19 requires antivirals. Considering the urgency to identify effective antiviral drugs, and the usually lengthy process involved in approving candidate drugs for human use, it is beneficial to identify existing drugs already approved for use in humans that can be repurposed as safe and effective therapeutics for treating COVID-19 infections, and which may also be useful as lead molecules for novel drug development. Viral proteases have been successfully targeted for the development of antiviral drugs against human immunodeficiency virus-1 (HIV-1) and hepatitis C virus (HCV), and other viruses, and it has been suggested that some of these drugs may also be inhibitors of COVID-19 proteases and virus replication. Antivirals are particularly crucial to treat infected people during the period of time that an effective vaccine is being deployed. Antivirals can also potentially be used as prophylactic drugs to protect people from viral infection.

### SUMMARY

Some embodiments of the present disclosure are directed to a composition for treating a patient infected with a coronavirus including one or more inhibitors of SARS-CoV-2 RNA-dependent RNA polymerase (RdRp) and one or more inhibitors of SARS-CoV-2 papain-like protease (PL^{pro}) that are synergistic with the one or more inhibitors for inhibiting SARS-CoV-2 RdRp. In some embodiments, the SARS-CoV-2 RdRp inhibitors include remdesivir, favipiravir, molnupiravir (MK-4482/EIDD-2801), functional equivalents thereof, or combinations thereof. In some embodiments, the SARS-CoV-2 PL^{pro} inhibitors also inhibit hepatitis C virus NS3/4A protease. In some embodiments, the SARS-CoV-2 PL^{pro} inhibitors include grazoprevir, vaniprevir, paritaprevir, GRL0617, Compound 6 (an analog of GRL0617), functional equivalents thereof, or combinations thereof. In some embodiments, the composition includes one or more inhibitors of viral proteins, their complexes, or combinations thereof, that are generated by SARS-CoV-2 PL^{pro} and/or main protease (M^{pro}) cleavage. In some embodiments, the composition includes inhibitors of non-structural proteins NSP4, NSP6, NSP7, NSP8, NSP9, NSP10, NSP11, NSP12 (RNA polymerase), NSP13, NSP14, NSP15, NSP16, complexes formed between these non-structural proteins, or combinations thereof. In some embodiments, the composition includes one or more additional active ingredients. In some embodiments, one or more pharmaceutically acceptable adjuvants, diluents, excipients, carriers, or combinations thereof. In some embodiments, the composition includes about 5 to about 10-fold less SARS-CoV-2 RdRp inhibitor than the recommended dosage of the SARS-CoV-2 RdRp inhibitor. In some embodiments, the composition includes about 5 to about 10-fold less SARS-CoV-2 PL^{pro} inhibitor than the recommended dosage of the SARS-CoV-2 PL^{pro} inhibitor.

Some embodiments of the present disclosure are directed to a method of treating a patient infected with SARS-CoV-2 including identifying a SARS-CoV-2 infection in the patient and administering an effective amount of a composition to the patient. In some embodiments, the composition includes one or more inhibitors of SARS-CoV-2 RdRp and one or more inhibitors of SARS-CoV-2 PL^{pro} and that are synergistic with the one or more inhibitors for inhibiting SARS-CoV-2 RdRp. In some embodiments, administering an effective amount of a composition to the patient includes administering to the patient about 5 to about 10-fold less SARS-CoV-2 RdRp inhibitor than the recommended dosage of the SARS-CoV-2 RdRp inhibitor. In some embodiments, administering an effective amount of a composition to the patient includes administering to the patient about 5 to about 10-fold less SARS-CoV-2 PL^{pro} inhibitor than the recommended dosage of the SARS-CoV-2 PL^{pro} inhibitor.

In some embodiments, SARS-CoV-2 RdRp inhibitors include remdesivir, favipiravir, molnupiravir (MK-4482/EIDD-2801), functional equivalents thereof, or combinations thereof. In some embodiments, the SARS-CoV-2 PL^{pro} inhibitors also inhibit hepatitis C virus NS3/4A protease. In some embodiments, the SARS-CoV-2 PL^{pro} inhibitors include grazoprevir, vaniprevir, paritaprevir, GRL0617, Compound 6 (an analog of GRL0617), functional equivalents thereof, or combinations thereof. In some embodiments, the composition includes one or more inhibitors of viral proteins, their complexes, or combinations thereof, that are generated by SARS-CoV-2 PL^{pro} and/or M^{pro} cleavage. In some embodiments, the composition includes inhibitors of non-structural proteins NSP4, NSP6, NSP7, NSP8, NSP9, NSP10, NSP11, NSP12 (RNA polymerase), NSP13, NSP14, NSP15, NSP16, complexes formed between these non-structural proteins, or combinations thereof. In some embodiments, the composition includes one or more additional active ingredients. In some embodiments, the one or more pharmaceutically acceptable adjuvants, diluents, excipients, carriers, or combinations thereof.

Some embodiments of the present disclosure include a method of treating a patient infected with SARS-CoV-2 including identifying a SARS-CoV-2 infection in the patient and administering to the patient an effective amount of an inhibitor of SARS-CoV-2 RdRp and an effective amount of an inhibitor of SARS-CoV-2 PL^{pro} that is also synergistic with the SARS-CoV-2 RdRp inhibitor. In some embodiments, the SARS-CoV-2 PL^{pro} inhibitor is paritaprevir. In some embodiments, the effective amount of the SARS-CoV-2 RdRp inhibitor is about 5 to about 10-fold less SARS-CoV-2 RdRp inhibitor than the recommended dosage of the SARS-CoV-2 RdRp inhibitor. In some embodiments, the effective amount of the SARS-CoV-2 PL^{pro} inhibitor is about 5 to about 10-fold less SARS-CoV-2 PL^{pro} inhibitor than the recommended dosage of the SARS-CoV-2 PL^{pro} inhibitor. In some embodiments, the SARS-CoV-2 polymerase inhibitor is orally ingestible by the patient.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A is a schematic drawing of the protein structure of SARS-CoV-2 main protease (M^{pro});

FIG. 1B is a schematic drawing of the protein structure of hepatitis C virus (HCV) serine protease;

FIG. 1C is a schematic drawing of a superimposition of the backbone structures of M^{pro} and HCV serine protease;

FIG. 2A portrays graphs of virtual docking study data identifying potential inhibition of M^{pro} by HCV serine protease inhibitors;

FIG. 2B portrays graphs of 1D ¹H-NMR assay data identifying inhibition of M^{pro} by HCV serine protease inhibitors;

FIG. 2C portrays graphs of virtual docking study data identifying potential inhibition of SARS-CoV-2 papain-like protease (PL^{pro}) by HCV serine protease inhibitors;

FIG. 2D portrays graphs of Fluorescence Resonance Energy Transfer (FRET) assay data identifying inhibition of PL^{pro} by HCV serine protease inhibitors;

FIG. 3A portrays M^{pro} and PL^{pro} inhibition results of HCV serine protease inhibitors, as well as synergy results between HCV serine protease inhibitors and remdesivir;

FIG. 3B portrays M^{pro} and PL^{pro} inhibition results of GC-376 and Compound 6;

FIG. 3C portrays graphs confirming synergy between PL^{pro} inhibitors and the SARS-CoV-2 RdRp inhibitor, remdesivir; and

FIG. 4 is a chart of a method of treating a patient infected with SARS-CoV-2 according to some embodiments of the present disclosure.

### DETAILED DESCRIPTION

Aspects of the present disclosure include a composition for treating a patient infected with a coronavirus. In some embodiments, the composition treats the infected patient by inhibiting the activity of the coronavirus, inhibiting replication of the coronavirus, or combinations thereof. As used herein, the term “inhibitor” is used to refer to absolute inhibitors of a given target, as well as strong inhibitors, e.g., having a ratio of initial velocity in the presence of inhibitor (vᵢ) to initial velocity in the absence of inhibitor (vᵢ₀) of less than about 90% as measured by Fluorescence Resonance Energy Transfer (FRET) assay.

In some embodiments, the coronavirus to be treated is Severe Acute Respiratory Syndrome virus (SARS), Middle East Respiratory Syndrome virus (MERS), or Corona Virus Infectious Disease 2019 virus (SARS-CoV-2 or COVID-19). In some embodiments, the composition includes one or more inhibitors for inhibiting coronavirus polymerases. In some embodiments, the composition includes one or more inhibitors for inhibiting SARS-CoV-2 polymerases. In some embodiments, the composition includes one or more inhibitors of SARS-CoV-2 RNA-dependent RNA polymerase (RdRp). In some embodiments, the composition includes remdesivir, favipiravir, molnupiravir (MK-4482/EIDD-2801), functional equivalents thereof, or combinations thereof. In some embodiments, the SARS-CoV-2 polymerase inhibitor is orally ingestible by the patient.

In some embodiments, the composition includes one or more inhibitors for inhibiting coronavirus proteases. In some embodiments, the composition includes one or more inhibitors for inhibiting SARS-CoV-2 proteases. In some embodiments, the composition includes one or more inhibitors of SARS-CoV-2 papain-like protease (PL^{pro}). In some embodiments, the composition includes one or more inhibitors of SARS-CoV-2 main protease (M^{pro}). In some embodiments, the composition includes one or more inhibitors for inhibiting both M^{pro} and PL^{pro}. In some embodiments, the protease inhibitors are also effective for inhibiting hepatitis C virus (HCV) proteases. In some embodiments, the protease inhibitors also inhibit HCV NS3/4 protease. In some embodiments, the SARS-CoV-2 PL^{pro} inhibitors also inhibit HCV NS3/4A protease. In some embodiments, the SARS-CoV-2 M^{pro} inhibitors also inhibit HCV NS3/4A protease.

In some embodiments, the one or more inhibitors for inhibiting PL^{pro} are synergistic with one or more inhibitors for inhibiting SARS-CoV-2. In some embodiments, the one or more inhibitors for inhibiting PL^{pro} are synergistic with one or more inhibitors for inhibiting SARS-CoV-2 polymerases. In some embodiments, the SARS-CoV-2 PL^{pro} inhibitors are synergistic with the one or more inhibitors for inhibiting SARS-CoV-2 RdRp. Without wishing to be bound by theory, molecules which inhibit PL^{pro} should be synergistic with molecules that inhibit SARS-CoV-2 polymerases, e.g., RdRp. Synergy between inhibitors, e.g., PL^{pro}, M^{pro}, and polymerase inhibitors, was predicted since M^{pro} processes a viral polyprotein to generate three viral polymerase subunits, as will be discussed in greater detail below. In some embodiments, the plasma concentration of the one or more protease inhibitors in the patient is made to be between about of 0.5 to 50 micromolar. In some embodiments, the plasma concentration of the one or more protease inhibitors in the patient is made to be between about of 0.5 to 1 micromolar. In some embodiments, the plasma concentration of the one or more protease inhibitors in the patient is made to be about 1 micromolar. In some embodiments, the SARS-CoV-2 PL^{pro} inhibitors include grazoprevir, vaniprevir, paritaprevir, GRL0617, Compound 6 (presented below, an analog of GRL0617), functional equivalents thereof, or combinations thereof.

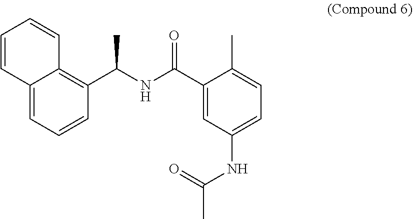 [SMILES: CC(=O)Nc1ccc(C)c(C(=O)N[C@H](C)c2cccc3ccccc23)c1]

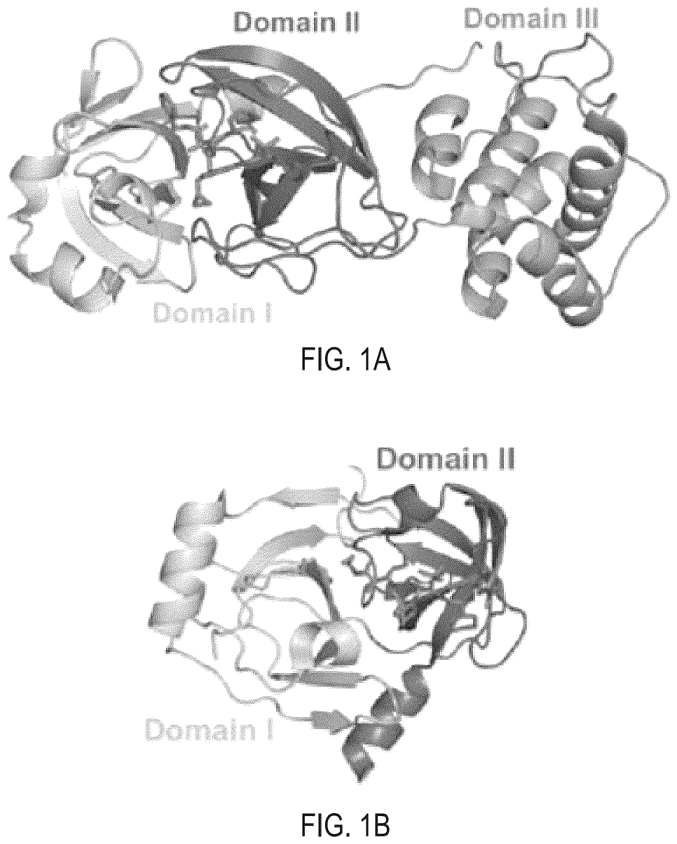

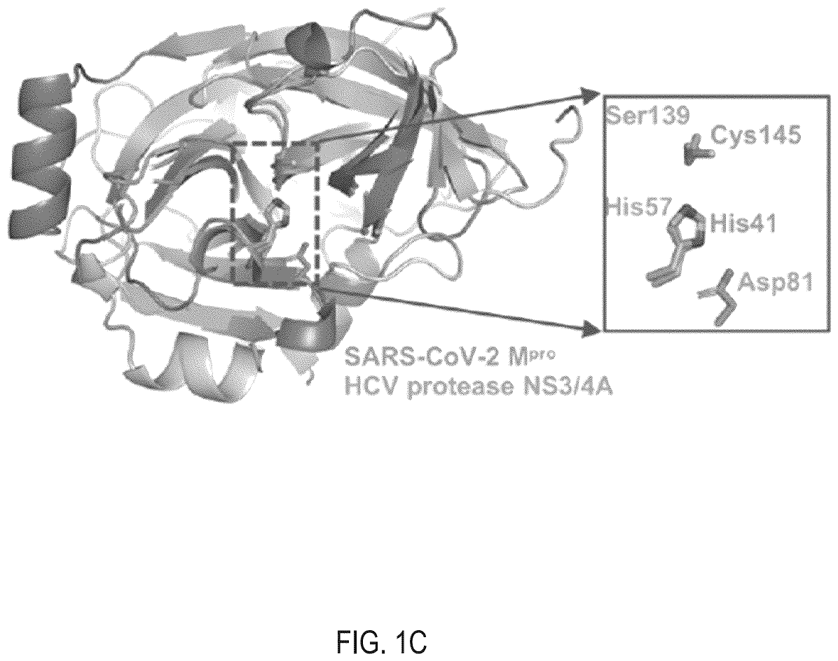

To provide antiviral drugs that can be rapidly deployed to combat the COVID-19 pandemic, studies were carried out to identify currently available drugs that could potentially be repurposed as inhibitors of SARS-CoV-2. Instead of screening libraries of current drugs, a search was performed based on the striking similarity of the substrate binding clefts of the SARS-CoV-2 M^{pro} and HCV NS3/4A proteases. Referring now to FIG. 1A, M^{pro} is a 67.6 kDa homodimeric cysteine protease with three domains. Domains I and II adopt a double β-barrel fold, with the substrate binding site located in a shallow cleft between two antiparallel β-barrels of domain I and II. Without wishing to be bound by theory, C-terminal domain III stabilizes the homodimer. The fold architecture of domains I and II are similar to picornavirus cysteine proteases and chymotrypsin serine proteases. The three-dimensional structural similarity search of the Protein Data Bank using the DALI program, with domains I and II (excluding domain III) of the SARS-CoV-2 M^{pro} as the three-dimensional structural query, identified several proteases, including the HCV NS3/4A serine protease, as structurally similar. These two enzymes have a structural similarity Z score of +8.4, and overall backbone root-mean-squared deviation for structurally similar regions of ˜3.1 Å.

Referring now to FIG. 1B, the HCV serine protease also has a double β-barrel fold, with relative orientations similar to those of the SARS-CoV-2 M^{pro} cysteine protease, and a substrate binding site located in a shallow cleft between its two six- to eight-stranded antiparallel 3-barrels. Superimposition of the backbone structures of these two proteases results in superimposition of their substrate binding cleft and their active-site catalytic residues, His41/Cys145 of the SARS-CoV-2 M^{pro} cysteine protease and His57/Ser139 of the HCV NS3/4A serine protease (see FIG. 1C). Because of these structural similarities, it was reasoned that some HCV protease inhibitors might bind into the substrate binding cleft of the SARS-CoV-2 M^{pro} and inhibit virus replication.

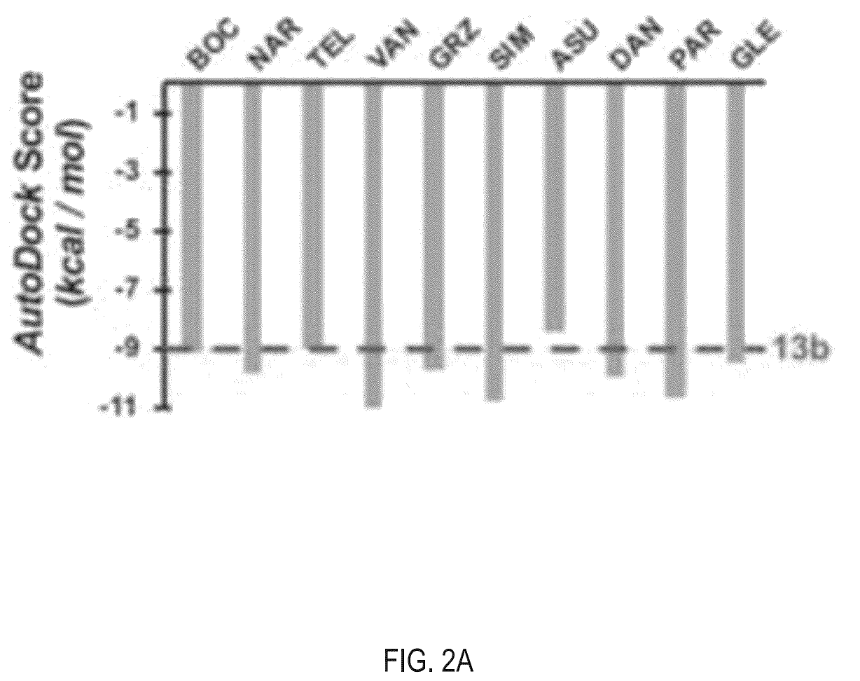

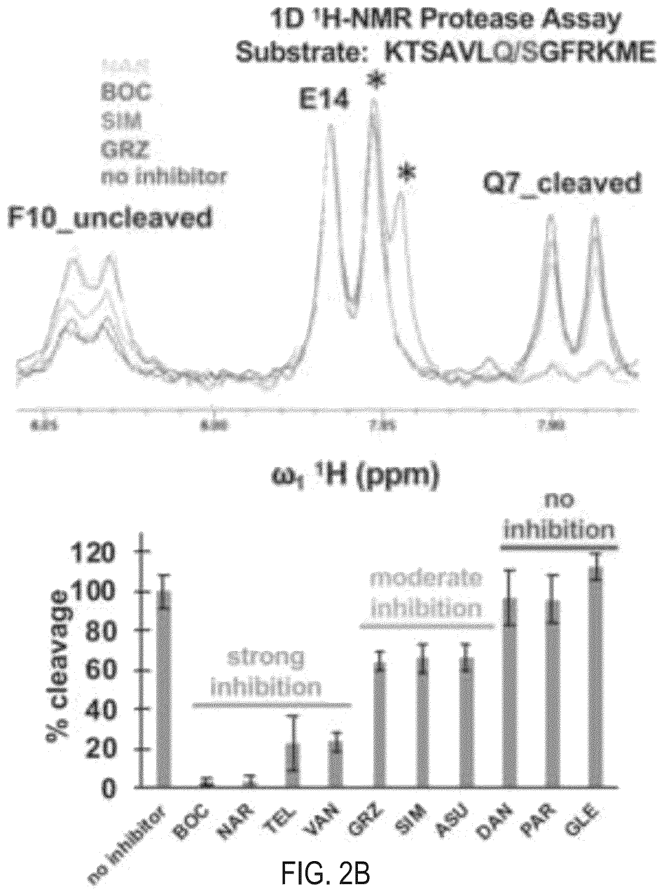

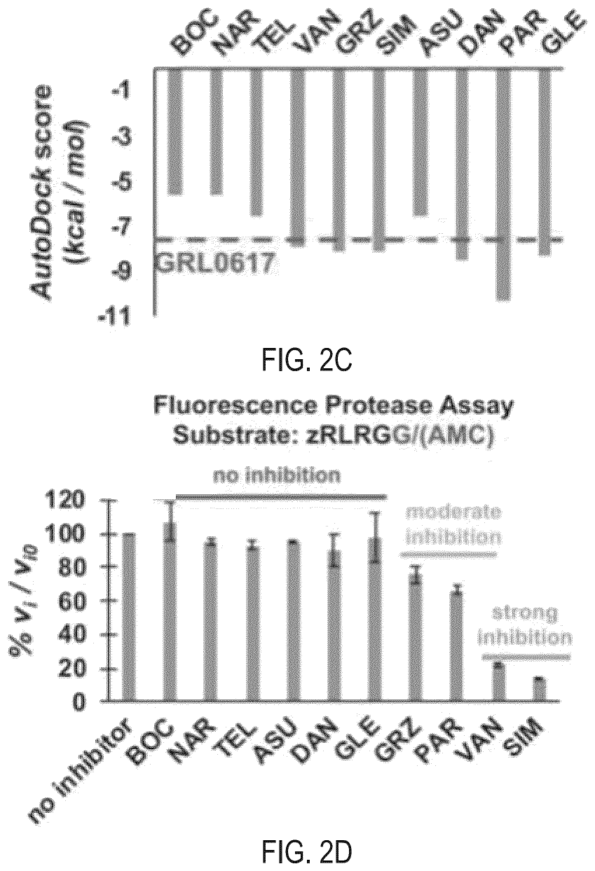

Referring now to FIG. 2A, virtual docking experiments were carried out on ten HCV protease inhibitors, which showed they can be docked snuggly into the substrate binding cleft of M^{pro}, and hence would have the potential to inhibit binding of the M^{pro} substrate, thereby inhibiting proteolytic cleavage of the substrate. Four of these HCV drugs, boceprevir, narlaprevir, telaprevir, and vaniprevir are relatively strong inhibitors of SARS-CoV-2 M^{pro} protease activity (IC₅₀ of 2 to ˜20 μM), and three other HCV drugs, grazoprevir, simeprevir and asunaprevir moderately inhibit M^{pro} activity. For boceprevir and narlaprevir, these predicted docking poses are consistent with the subsequently determined X-ray crystal structures; for telaprevir some predicted binding poses are also similar to the corresponding crystal structure. Boceprevir, narlaprevir, and telaprevir are a-keto amides, which can form a covalent bond with the active site Cys thiol of M^{pro}. Without wishing to be bound by theory, the other four HCV drugs are non-covalent inhibitors of the M^{pro} protease and may not form a covalent bond with the active site Cys thiol. A total of eight HCV drugs inhibit virus replication in Vero and/or human 293T cells expressing the ACE2 receptor.

Referring now to FIG. 2B, 1D ¹H-NMR assay was used to identify M^{pro} inhibition, which confirmed that boceprevir, narlaprevir, and telaprevir inhibit M^{pro}. In the NMR assay, vaniprevir also had inhibitory activity comparable to telaprevir, and grazoprevir and simeprevir were moderate inhibitors of M^{pro}.

Again, without wishing to be bound by theory, aside from both being Cys proteases, the active site of PL^{pro} does not share structural similarity with the HCV 3/4A or M^{pro} proteases. However, it is possible that some of these HCV protease inhibitors can also bind into the active site of PL^{pro}. Accordingly, referring now to FIG. 2C, virtual docking studies were carried out of the ten HCV drugs into the substrate binding cleft of PL^{pro}, using protocols similar to those developed in virtual docking studies with M^{pro}. The PL^{pro} inhibitor GRL0617, for which a crystal structure bound to PL^{pro} is available in the PDB, was used to assess the docking protocol and to determine a reference AutoDock score of −7.54 kcal/mol. The scores of docking poses for HCV drugs ranged from −5.56 kcal/mole for boceprevir and narlaprevir, to much more favorable values of <−8 kcal/mol for others including vaniprevir, grazoprevir, simeprevir, and paritaprevir. Without wishing to be bound by theory, none of these four inhibitors may form covalent bonds with the active-site Cys residue of PL^{pro}. Vaniprevir was a good inhibitor of both M^{pro} and PL^{pro}. These proof-of concept docking studies suggested that, surprisingly, some HCV protease inhibitors may bind in the active sites of both M^{pro} and PL^{pro}.

Referring now to FIG. 2D, based on these docking results, it was anticipated that several HCV protease inhibitors might effectively inhibit PL^{pro} protease activity. To test this, fluorescence assays of PL^{pro} inhibition were carried out, using the substrate zRLRGG/AMC (z-carboxybenzyl; AMC-7-Amino-4-methylcoumarin) including a natural canonical PL^{pro} protease recognition site (XLXGG). In this assay, the fluorescence of the AMC group was quenched when covalently attached to the peptide and increases upon proteolytic cleavage. Of the HCV drugs tested, vaniprevir, simeprevir, paritaprevir, and grazoprevir did inhibit PL^{pro} protease activity. Hence, vaniprevir, simeprevir, and grazoprevir inhibited both M^{pro} and PL^{pro}, while paritaprevir inhibited PL^{pro}, but not M^{pro}. Under these assay conditions, vaniprevir and simeprevir were more effective PL^{pro} inhibitors than grazoprevir or paritaprevir.

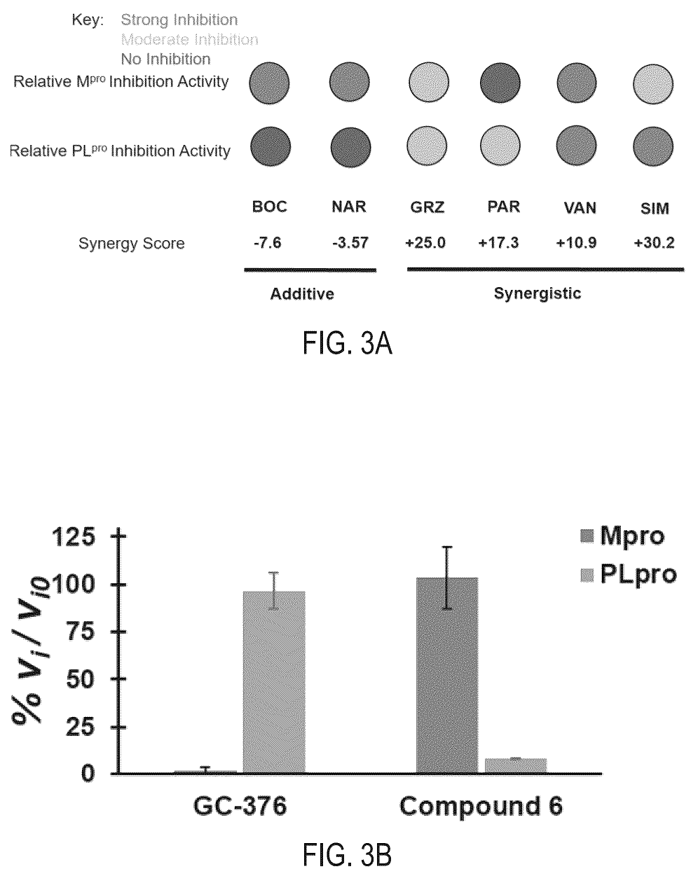

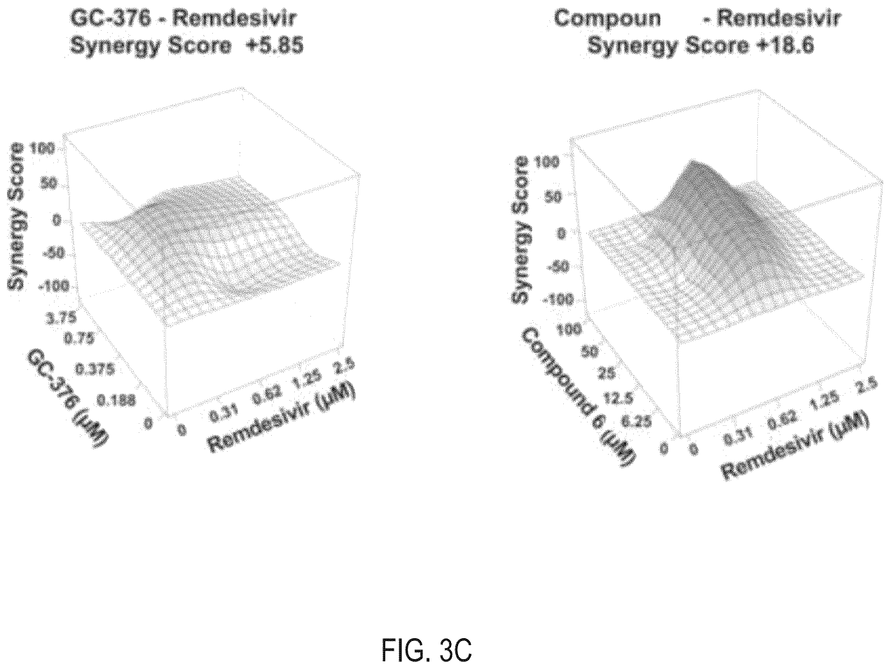

Referring now to FIG. 3A, viral replication assays using combinations of drugs allowed assessment as to whether the interactions between HCV drugs and remdesivir are additive or synergistic, i.e., resulting in an inhibition of virus replication that is greater than the sum of the inhibitory activities of the HCV drug and remdesivir. Surprisingly, as can be seen in Table 1 below, these inhibitory effects were additive or synergistic depending on which HCV drug is used to inhibit virus replication. Without wishing to be bound by theory, HCV drugs that inhibited PL^{pro}, strongly or moderately, synergized with remdesivir to inhibit SARS-CoV-2 replication in Vero and 293T cells. These HCV drugs included simeprevir, vaniprevir, grazoprevir, and paritaprevir. Inhibition of PL^{pro} alone appeared to be sufficient for synergy with remdesivir, based on a synergy assay with Compound 6 that specifically inhibits PL^{pro} but not M^{pro} (see FIGS. 3B-3C, in comparison with M^{pro} inhibitor GC-376). On the other hand, it was shown that the HCV drugs such as boceprevir and narlaprevir that inhibit M^{pro} have an additive rather than a synergistic interaction with remdesivir in inhibiting SARS-CoV-2 replication. Particularly interesting in this data set was paritaprevir, which exhibited the strongest potency in the human cell assay (IC₅₀=0.55 μM), strong synergy with remdesivir (synergy score+17.3), and low cytotoxicity (CC₅₀>100 μM) in both the Vero and human cell assays.

TABLE 1
Comparison of enzyme inhibition and viral inhibition activities of HCV protease
inhibitors. (a) Strong inhibition corresponds to an estimated IC50 < about
20 μM, moderate to an estimated IC50 in the range 20-50 μM, and none indicates
no observed inhibition of protease activity at drug concentrations of 50 μM.

| | | | | | Vero | HEK | | |
|---|---|---|---|---|---|---|---|---|
| | Relative | Relative | | | E6 | 293T | | |
| | M^{pro} | PL^{pro} | | | Cells | Cells | Synergy | |
| | Inhibition | Inhibition | IC₅₀ | CC₅₀ | IC₅₀ | CC₅₀ | Score | |
| BOC | strong | none | 19.6 | >50 | 5.4 | >50 | −7.6 | additive |
| NAR | Strong | none | 7.7 | >20 | 15.0 | 72.0 | −3.6 | additive |
| SIM | moderate | strong | 4.2 | 2.1 | 2.3 | >50 | +30.2 | synergistic |
| VAN | strong | strong | 6.2 | 4.3 | 3.0 | >50 | +10.9 | synergistic |
| PAR | none | moderate | 6.0 | >100 | 0.55 | >100 | +17.3 | synergistic |
| GRZ | moderate | moderate | 10.8 | >50 | 16.7 | >50 | +25.0 | synergistic |

Without wishing to be bound by theory, one mechanism through which PL^{pro} inhibition may result in synergy with remdesivir involves the role of PL^{pro} in the formation of replication organelles (double membrane vesicles, DMVs). Studies of DMV formation by SARS-CoV NSP3, NSP4, and NSP6 proteins demonstrate a use of all three proteins, and for a catalytically active PL^{pro} NSP3 construct. HCV drugs that inhibit PL^{pro} in infected cells should therefore inhibit formation of DMVs that are used for polymerase function, reducing the amount of functional viral RNA polymerases, and hence reducing the amount of remdesivir for inhibition of virus replication. This hypothetical mechanism could explain why drugs that inhibit PL^{pro}, e.g., simeprevir, vaniprevir, paritaprevir, and grazoprevir, acted synergistically with remdesivir. In contrast, M^{pro} inhibitors are expected to reduce the amount of the three subunits, NSP7, NSP8 and NSP12, of the viral polymerase in infected cells. However, the reduction in the amounts of these polymerase subunits might not reduce the level of the viral polymerase sufficiently to exhibit synergy with remdesivir if there is relatively large pool of these subunits in infected cells. In addition, synergy with remdesivir could also involve other viral or host targets of these protease inhibitors.

Without wishing to be bound by theory, synergy between PL^{pro} and viral polymerase inhibitors could also involve other viral or host targets of these protease inhibitors. Removal of ISG15 from viral or host proteins by PL^{pro} could potentially restore their functions, and inhibition of the de-ISGlyation function of PL^{pro} could provide another mechanism for synergy between inhibitors of PL^{pro} and inhibitors of other viral or host protein functions, including remdesivir. In particular, through this mechanism PL^{pro} inhibitors could potentially also synergize with inhibitors of other viral proteins or host proteins independent of inhibition of RdRp.

In some embodiments, the composition includes one or more inhibitors of viral proteins, their complexes, or combinations thereof, that are generated by SARS-CoV-2 PL^{pro} and/or M^{pro} cleavage. In some embodiments, the composition includes inhibitors of non-structural proteins NSP4, NSP6, NSP7, NSP8, NSP9, NSP10, NSP11, NSP12 (RNA polymerase), NSP13, NSP14, NSP15, NSP16, complexes formed between these non-structural proteins, or combinations thereof. In some embodiments, the composition includes one or more additional inhibitors, the additional inhibitors configured to inhibit non-M^{pro} targets, non-PL^{pro}, other targets, etc., or combinations thereof. In some embodiments, the composition includes one or more additional active ingredients. In some embodiments, the composition includes one or more pharmaceutically acceptable adjuvants, diluents, excipients, carriers, or combinations thereof.

In some embodiments, the composition includes a combination of a SARS-CoV-2 RdRp inhibitor and a SARS-CoV-2 PL^{pro} inhibitor. In some embodiments, the composition includes amounts of a SARS-CoV-2 RdRp inhibitor and a SARS-CoV-2 PL^{pro} inhibitor to be administered separately, e.g., in separate orally administered doses. In some embodiments, the composition includes about 5 to about 10-fold less SARS-CoV-2 RdRp inhibitor than the recommended dosage of the SARS-CoV-2 RdRp inhibitor. By way of example, the recommended dosage of remdesivir to an adult infected with SARS-CoV-2 is 200 mg on Day 1 of treatment, and 100 mg on Day 2 and each successive day of treatment. Thus, in an exemplary embodiment, a patient infected with SARS-CoV-2 is administered the composition so as to receive 20 mg and 40 mg of the SARS-CoV-2 RdRp inhibitor on Day 1 of treatment, between about 10 mg and about 20 mg of the SARS-CoV-2 RdRp inhibitor on Day 2 of treatment, etc. In some embodiments, the composition includes about 5 to about 10-fold less SARS-CoV-2 PL^{pro} inhibitor than the recommended dosage of the SARS-CoV-2 PL^{pro} inhibitor.

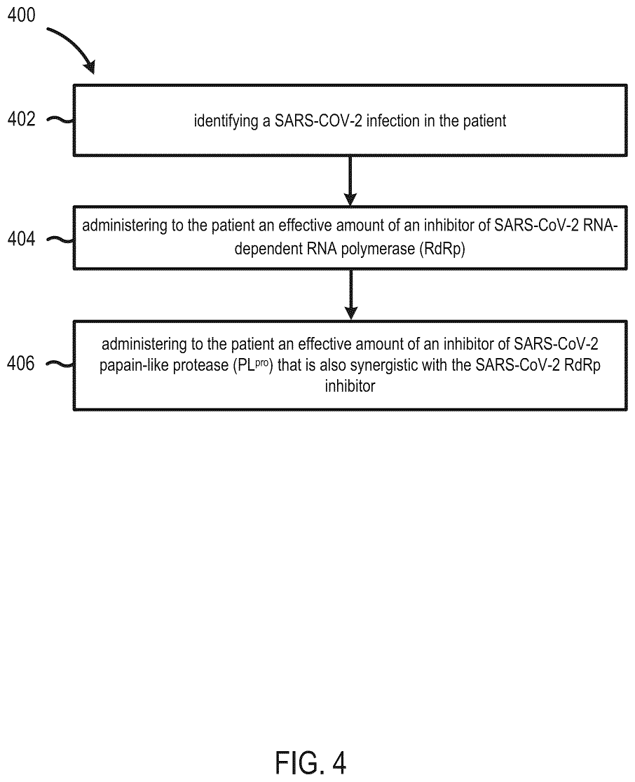

Referring now to FIG. 4, some aspects of the present disclosure are directed to a method 400 of treating a patient infected with a coronavirus, e.g., SARS-CoV-2. At 402, a coronavirus infection, e.g., a SARS-CoV-2 infection, is identified in the patient. At 404, an effective amount of an inhibitor of SARS-CoV-2 polymerase, e.g., RdRp, is administered to a patient. In some embodiments, administering 404 includes administering to the patient about 5 to about 10-fold less SARS-CoV-2 RdRp inhibitor than the recommended dosage of the SARS-CoV-2 RdRp inhibitor. At 406, an effective amount of an inhibitor of SARS-CoV-2 protease, e.g., PL^{pro} is administered to the patient. In some embodiments, administering 406 includes administering to the patient about 5 to about 10-fold less SARS-CoV-2 PL^{pro} inhibitor than the recommended dosage of the SARS-CoV-2 PL^{pro} inhibitor. As discussed above, in some embodiments, the SARS-CoV-2 PL^{pro} inhibitor is synergistic with the SARS-CoV-2 RdRp inhibitor. In some embodiments, the SARS-CoV-2 RdRp inhibitor and the SARS-CoV-2 PL^{pro} inhibitor are administered as part of the same composition.

As discussed above, in some embodiments, the SARS-CoV-2 polymerase inhibitor includes remdesivir, favipiravir, molnupiravir (MK-4482/EIDD-2801), functional equivalents thereof, or combinations thereof. In some embodiments, the SARS-CoV-2 polymerase inhibitor is orally ingestible by the patient. In some embodiments, the SARS-CoV-2 protease inhibitor also inhibits hepatitis C virus NS3/4A protease. In some embodiments, the SARS-CoV-2 protease inhibitor includes grazoprevir, vaniprevir, paritaprevir, GRL0617, Compound 6, functional equivalents thereof, or combinations thereof. In some embodiments, the composition includes one or more inhibitors of viral proteins, their complexes, or combinations thereof, that are generated by SARS-CoV-2 PL^{pro} and/or M^{pro} cleavage. In some embodiments, one or more inhibitors of non-structural proteins NSP4, NSP6, NSP7, NSP8, NSP9, NSP10, NSP11, NSP12 (RNA polymerase), NSP13, NSP14, NSP15, NSP16, complexes formed between these non-structural proteins, or combinations thereof are also administered. In some embodiments, one or more additional active ingredients are also administered. In some embodiments, one or more pharmaceutically acceptable adjuvants, diluents, excipients, carriers, or combinations thereof are also administered.

### Methods

The free open source AutoDock suite was used. AutoDockTools was used for coordinate preparation, docking, and analysis of results. The computational docking program AutoDock v4.2.6. is based on an empirical free energy force field and uses a search method based on Lamarckian genetic algorithm. Target protein coordinates were obtained from SARS-CoV-2 Mpro X-ray crystal structure (PDB id 6Y2G), and structural water was removed. Three-dimensional coordinates for ligand molecules were obtained from PDB or from chemical structure databases, ChemSpider and DrugBank. Protein and ligand coordinates were then prepared using AutoDockTools; polar hydrogens were added to protein structures, and Gasteiger-Marsili empirical atomic partial charges were added to ligands. Torsional degrees of freedom (dihedral angles) were identified for each ligand. These data and parameters for each protein and ligand were saved as individual PDBQT files. In these studies, ligand dihedral angles were allowed to vary (except where stated otherwise), and all protein dihedral angles were kept rigid. The program Autogrid was used to prepare affinity maps for all atom types in the receptor and ligands. A grid of 56, 40 and 48 points in x, y and z direction, with a grid spacing of 0.375 Å was used to compute electrostatic maps. The grid center was placed on the center of inhibitor 13b molecule in its complex with M^{pro} (PDB id 6Y2G). The Lamarckian genetic algorithm (LGA) method was used for sampling ligand binding conformation, with the following LGA parameters: 150 individuals in population; 2,500,000 energy evaluations; 27,000 maximum number of generations; and with mutation and crossover rates of 0.02 and 0.08, respectively. A maximum of 300 iterations per local search was used. The calculations were repeated for 100 docking simulations for each complex. For a comparative analysis, docking simulations of α-ketoamide inhibitor 13b were also performed using the same protocol used for docking the protease inhibitor drugs. All docking simulations were analyzed using the AutoDockTools. Atomic coordinates for best scoring conformation obtained in each docking simulation, for each drug-protein complex, were saved in PDB format for analysis. Without wishing to be bound by theory, because the experimentally determined pose is often not the one with the lowest docking energy, but rather is found among other highly ranked poses, other low energy poses were also examined. These protein-ligand complexes were analyzed in detailed using open source PyMol molecular visualization tool and fully automated Protein-Ligand Interaction Profiler.

PL^{pro} inhibitor complexes in the PDB revealed that the BL2 loop present at the entrance of active site adopts significantly different conformations depending on the size of the inhibitor bound to the PL^{pro}. This plasticity in the BL2 loop suggests an induced fit mechanism of ligand binding to PL^{pro} active site. To avoid a closed conformation of the BL2 loop found in protein ligand complex, SARS-CoV-2 PL^{pro} X-ray crystal structure in its apo form (PDB id 6W9C) was chosen as the target to dock HCV protease inhibitors. The docking protocol was same as above, except a larger grid of size 56, 56, and 58 points in the x, y and z direction respectively was used to compute electrostatic maps for PL^{pro} target. For a comparative analysis, docking simulations of PL^{pro} inhibitor GRL0617 (PDB id 7CJM) were also performed using the same protocol.

The full-length SARS-CoV-2 M^{pro} gene corresponding to residues 3264 to 3567 of the SARS-CoV-2 replicase polyprotein 1a [Uniprot id PODTD1 (R1AB_SARS2)]; was obtained from GenScript USA Inc. in pGEX-6β-1 vector, as previously described. This expression vector is designated GTM_COV2_NSP5_001. This plasmid, which expresses SARS-CoV-2 M^{pro} as a self-cleaving (using its native cleavage site) GST-fusion, was transformed into competent E. coli BL21(DE3) cells. A single colony was picked and inoculated in 2 mL LB supplemented with 0.1 mg/ml Ampicillin at 37° C. and 225 rpm. The 2 mL inoculum was added to 1 L LB broth with 0.1 mg/mL ampicillin. The cells were allowed to grow to an optical density of 0.6 at 600 nm at 37° C. and 225 rpm and were induced with 1 mM IPTG. The induced cells were incubated overnight at 18° C. and 225 rpm. The cells were harvested and resuspended in lysis buffer (20 mM Tris pH 8.0, 300 mM NaCl) and then lysed by sonication. The cell debris was removed by centrifugation at 14,000 rpm for 40 mins. The supernatant was added to a Ni-NTA column preequilibrated with loading buffer (20 mM Tris pH 8.0, 300 mM NaCl, 10 mM imidazole), and bound proteins were eluted with 20 mM Tris pH 8.0, including 100 mM NaCl and 100 mM imidazole. The elution fractions that included M^{pro} were buffer exchanged (20 mM Tris pH 8.0, 100 mM NaCl, 1 mM EDTA, 1 mM DTT) and concentrated using a swinging bucket centrifuge at 4000 rpm. The final M^{pro} concentration was 23.3 μM, determined by absorbance at 280 nm using a calculated extinction coefficient of 33,640 M⁻¹ cm⁻¹. Homogeneity was validated by SDS-PAGE (>95% homogeneous), and the construct was validated by MALDI-TOF mass spectrometry. The enzymatic activity of freshly purified M^{pro} was measured using Michealis Menten equation best fit values of K_{M} and Vₘₐₓ; these were 55.5 M and 0.018 μM/sec, respectively. The calculated k_{cat} was 1.80 sec⁻¹, and k_{cat}/K_{M} was 32,400 sec⁻¹ M⁻¹.

M^{pro} was observed to be unusually sensitive to active site Cys oxidation and thus special care was taken in preparing samples, maintaining them under reducing conditions, and checking samples for time-dependent loss of activity over the course of enzyme activity and drug inhibition measurements. Purified samples of M^{pro} were prepared in 20 mM TRIS buffer, pH 8.0, including 100 mM NaCl, 1 mM EDTA, and 1 mM DTT, flash frozen in 50 μL aliquots, and stored at −80 deg. For enzyme assays, freshly thawed enzyme aliquots were prepared in buffers including 3 mM TCEP and assayed for specific activity at the beginning and end of the data collection session, or back-to-back with each measurement, in order to avoid spurious results due to enzyme inactivation during a measurement session.

The proteolysis of substrate KTSAVLQ/SGFRKME was studied using FRET and NMR assays. Fluorescence studies were carried out using an Infinite M1000 TECAN plate reader, with 3 mm path lengths, and Magellan™ software. NMR assays were carried out using Bruker Avance II 600 MHz NMR spectrometer system. For the FRET assay the substrate was Dabsyl-KTSAVLQ/SGFRKME-Edans, labeled with Dabcyl and Edans FRET pair on the N and C-termini of the peptide, respectively, as described elsewhere. The NMR assay used the same peptide substrate without the fluorescence dyes attached. Both labeled and unlabeled peptide substrates were obtained from GenScript USA, Inc.

As part of the initial development of the FRET assay for M^{pro} activity, catalysis rates were assessed over the range of 1 nM to 200 nM enzyme concentration, with the fluorophore-labeled peptide substrate. Significant proteolytic activity was observed over this whole range. As rates of hydrolysis at enzyme concentrations >˜50 nM are quite fast, the concentration of 10 nM was selected in order to provide the most accurate kinetic data. Other FRET-based assays of M^{pro} activity have been reported using enzyme concentrations ranging from 20 nM to 500 nM. However, it is well established that the kinetic properties of SARS-CoV M^{pro} are significantly influenced by the construct and assay conditions utilized. For SARS-CoV M^{pro}, the monomeric form is inactive and dimerization is present for its enzymatic activity. Reports of the dimer dissociation constant K_{d} for SARS-CoV M^{pro} range from 1 nM to 200 μM; these dimer dissociation constants are also very sensitive to the presence of non-native N- or C-terminal residues. The dimer dissociation constant K_{d} for a similar construct of SARS-CoV-2 M^{pro} has been reported to be ˜2.5 μM. Hence, the fraction of active enzyme present at 10 nM concentration is expected to be quite low. However, other groups have also reported SARS-CoV-2 M^{pro} enzyme activity assays at 20 nM enzyme concentration. Assuming the substrate binds more tightly to the dimer than to the monomer, the thermodynamic equilibrium between monomer and dimer would be expected to be shifted by the substrate, resulting in the observed protease activity even at enzyme concentrations well below the homodimer K_{d}. Alternatively, the monomeric form of the SARS-CoV-2 M^{pro} may in fact have significant protease activity.

All M^{pro} protease assays were carried out in Reaction Buffer including 20 mM HEPES pH 6.5, 120 mM NaCl, 0.4 mM EDTA and 3 mM TCEP. Additionally, 1 mg/mL BSA was added to the buffer for FRET assays. For the FRET assay, 10 nM M^{pro} was incubated with M of HCV drugs. The reaction was initiated by addition of 20 μM FRET substrate and monitored for 2 hours on an Infinite M1000 TECAN plate reader, exciting at 360 nm and detecting donor emission at 460 nm. The initial velocity of the reaction was calculated as the slope obtained from linear fits of emission intensity vs time plots for the first 15 minutes of the reaction. All FRET data were analyzed and plotted for initial velocity on Microsoft Excel.

In the FRET assays, the percent proteolytic activity in the presence of each drug was calculated as a ratio of initial velocity in presence if inhibitor (vᵢ) to initial velocity in absence of inhibitor (vᵢ₀), i.e., vᵢ/vᵢ₀. A histogram plot of vᵢ/vᵢ₀ for each inhibitor was used to compare relative inhibition activities. All FRET proteolysis reaction curves were measured twice, and the uncertainty in vᵢ/vᵢ₀, estimated from the standard deviation of 2 independent measurements, is shown as error bars. Short (<3 min) time points exhibiting equilibration artifacts and were excluded from this analysis.

For IC₅₀ measurements of HCV inhibitors BOC, NAR and TEL, 10 nM of M^{pro} was incubated with a range of inhibitor concentrations in the same Reaction Buffer described above. The inhibitor concentration ranges were 0.1-200 μM for BOC, 0.1 to 100 μM for NAR, and 0.2 to 100 μM for TEL. The reaction was initiated by adding 20 μM FRET substrate and monitored for 1 hr. Each measurement was repeated three times. The percent inhibition at each inhibitor concentration was calculated as:

% Inhibition=100*(vᵢ−vᵢ₀)/vᵢₘₐₓ−v₀)

where vᵢ=initial velocity at a given inhibitor concentration, vᵢ₀=initial velocity in absence of inhibitor, and vᵢₘₐₓ=initial velocity at maximum inhibition. The percent inhibition was plotted as a function of inhibitor concentration to obtain a dose-response curve using Prism 8 (GraphPad Software) software. IC₅₀ was calculated from fitting to the equation:

% Inhibition=100*[Inhibitor]/(IC₅₀+[Inhibitor])

For several of the HCV drugs, intrinsic fluorescence of the drugs appears to compromise the accuracy of the FRET kinetic curves. Even though little or no inner filter effects were observed in these measurements, simple subtraction of the drug fluorescence from the final values of kinetic curves results in curves which do not match the final values for curves obtained in the absence of drugs, suggesting possible energy transfer between the drug and the FRET fluorophores on the peptide substrate.

The inner filter effect results from absorbance of the sample at the fluorescence excitation wavelength, attenuating fluorescence excitation. Boceprevir, narlaprevir, and telaprevir, which do not have significant intrinsic fluorescence but have similar extinction coefficients to the other drugs at 360 nm, did not present a problem for the assay, indicating that inner filter effects are not significant. The inner filter effect was experimentally assessed by measuring fluorescence and emission for the fluorescent dye diethylamino naphthalene sulfonate (DENS) over the range 0.01 to 1.0 O.D. units, at the excitation wavelength (360 nm), using the Infinite M1000 TECAN plate reader fluorimeter. These data show that in this system the inner filter effects are negligible for samples with total OD360<0.025, similar to results reported elsewhere. With the exception of the vaniprevir study, these reaction mixtures had OD360<0.025. Hence, under the conditions of these assays, most of the assay samples have no significant inner filter effect; only the vaniprevir assay had a small effect, which was appropriately corrected for.

For the M^{pro 1}H NMR proteolysis assay the reaction was performed at 100 nM M^{pro} in the same assay buffer described above, along with 5% D₂O and 50 μM HCV inhibitors dissolved in d₆-DMSO (for the control experiments where no inhibitor is added, the same quantity of d₆-DMSO was added). 50 μM of unlabeled peptide substrate was added and immediately transferred to a 5-mm NMR tube. The final volume of each reaction mixture was 600 μL. The NMR tube was quickly placed in a 600 MHz Bruker Avance II spectrometer equipped with a 5-mm TCI cryoprobe, equilibrated at 298 K. The homogeneity of the magnetic field was adjusted by gradient shimming on the z-axis and in each case an array of 24 1H experiments was acquired with 1D 1H NMR using excitation sculpting for water suppression. The probe had previously been tuned and matched with a sample of similar composition. The delay between initiation of the reaction and starting acquisition was ˜5 mins for most of the reaction conditions. The duration of each NMR experiment was also considered to obtain accurate time values. All ¹H spectra were acquired, processed, and analyzed in Bruker TopSpin 3.6.2 software. The regions of interest were integrated, and the values obtained were transferred for further analysis and plotting.

These ¹H NMR spectra were used to monitor the evolution of substrate and product as a function of time. Resonance assignments (discussed below) of the cleaved and uncleaved KTSAVLQ/SGFRKME peptide identified the amide H^{N} resonances that were monitored during the reaction. The H^{N} resonances for amino-acid residues Phe-10 (uncleaved) and Gln-7 (cleaved) were used to quantify substrate utilization and product formation, respectively, during the reaction. The H^{N} peak intensity of residue Glu-14, which did not shift upon cleavage, was monitored as an internal control. The percent substrate cleavage in the presence of inhibitors at 30 min was calculated as a ratio of the H^{N} resonance integrals of Gln-7 in presence of inhibitor to the corresponding resonance integral with no inhibitor.

Chemical shift assignments of backbone amide ¹H and ¹⁵N resonances in the 14-residue peptide KTSAVLQSGFRKME in 20 mM HEPES pH 6.5, 100 mM NaCl, 0.4 mM EDTA, 3 mM TCEP and 5% ²H₂O were determined at 298 K using 2D COSY, TOCSY, and ¹H-¹⁵N HSQC, along with 1D ¹H NMR experiments, and referenced to internal DSS. Backbone amide ¹H and ¹⁵N resonances were assigned for 12/14 and 10/14 residues in the uncleaved and cleaved peptide respectively. These assignments have been deposited in the BioMagResDB as BMRB entries 50568 and 50569, respectively.

SARS-CoV-2 PL^{pro} enzyme was provided as a generous gift by Prof John Hunt (Columbia University). The construct residues 1564 to 1881 of the SARS-CoV-2 replicase polyprotein 1a (Uniprot id PODTD1 (R1A_SARS2)) was produced in expression vector pET21_NESG with a C-terminal purification tag LEI-HHHHH. Homogeneity (>95%) was validated by SDS-PAGE. The fluorogenic substrate zRLRGG/AMC was obtained from Bachem. All PL^{pro} proteolysis assays were carried out in buffer including 50 mM HEPES, pH 7.5, 5 mM DTT, 1 mg/ml BSA. For the fluorescence assay, 20 nM of PL^{pro} was incubated with M of HCV drugs. 20 μM substrate was added and the reaction was monitored for 2 hours using the Infinite M1000 TECAN plate reader compatible with Magellan™ software with filters for excitation at 360 nm and emission at 460 nm. No anomalous fluorescence interactions were observed in this assay for any of the drugs. The PL^{pro} fluorescence assay was also assessed for inner filter effects. Inner filter effects were also not significant in this assay, except for the vaniprevir study for which appropriate corrections were made.

The data points for the first 10 mins of the proteolysis reaction progression curves were used to calculate the initial velocity (vᵢ) in the presence and absence of the inhibitor. The percent proteolytic activity in presence of each drug is calculate as a ratio of initial velocity in presence of inhibitor (vᵢ) to initial velocity in absence of inhibitor (vᵢ₀), i.e. vᵢ/vᵢ₀. A histogram plot of vᵢ/vᵢ₀ for each inhibitor was used to compare relative inhibition activities. All proteolysis reaction curves were measured twice, and the standard deviation in vᵢ/vᵢ₀ is shown as error bars.

Vero E6 (ATCC, CRL-1586) and 293T cells (ATCC, CRL-3216; kind gift of Dr. Viviana Simon), were maintained in DMEM (Corning) supplemented with 10% FB (Peak Serum) and Penicillin/Streptomycin (Corning) at 37° C. and 5% CO₂. hACE2-293T cells were generated for this study. Briefly, 293T cells were transduced with a lentiviral vector expressing human ACE2. Puromycin resistant cells with hACE2 surface expression were sorted after staining with AlexaFluor 647-conjugated goat anti-hACE2 antibodies. Cells were then single-cell-cloned and screened for their ability to support SARS-CoV-2 replication. All cell lines used in this study were regularly screened for mycoplasma contamination using the Universal Detection Kit (ATCC, 30-1012K). Cells were infected with SARS-CoV-2, isolate USA-WA1/2020 (BEI Resources NR-52281) under biosafety level 3 (BSL3) containment in accordance to the biosafety protocols developed by the Icahn School of Medicine at Mount Sinai. Viral stocks were grown in Vero E6 cells as previously described and were validated by genome sequencing.

2,000 Vero E6 or hACE2-293T cells were seeded into 96-well plates in DMEM (10% FBS) and incubated for 24 h at 37C, 5% CO₂. Two hours before infection, the medium was replaced with 100 μL of DMEM (2% FBS) including the compound of interest at concentrations 50% greater than those indicated, including a DMSO control. Plates were then transferred into the BSL3 facility and 100 PFU (MOI=0.025) was added in 50 μL of DMEM (2% FBS), bringing the final compound concentration to those indicated. Plates were then incubated for 48 h at 37° C. After infection, supernatants were removed and cells were fixed with 4% formaldehyde for 24 hours prior to being removed from the BSL3 facility. The cells were then immunostained for the viral NP protein [an in-house mAb 1C7, provided by Dr. Thomas Moran (MSSM)] with a DAPI counterstain. Infected cells (488 nM) and total cells (DAPI) were quantified using the Celigo (Nexcelcom) imaging cytometer. Infectivity was measured by the accumulation of viral NP protein in the nucleus of the Vero E6 cells (fluorescence accumulation). Percent infection was quantified as

[(Infected cells/Total cells)−Background]*100

and the DMSO control was then set to 100% infection for analysis. The IC₅₀ and IC₉₀ for each experiment were determined using the Prism (GraphPad Software) software. Cytotoxicity was also performed using the MTT assay (Roche), according to the manufacturer's instructions. Cytotoxicity was performed in uninfected VeroE6 cells with same compound dilutions and concurrent with viral replication assay. All assays were performed in biologically independent triplicates. Remdesivir was purchased from Medkoo Bioscience Inc. Time of addition experiments were performed using the same immunofluorescence-based assay with the following alterations: Vero E6 cells were infected with 8000 PFU (MOI of 2) of SARS-CoV-2, the drug was added at different times relative to infection as indicated, and the infection was ended by fixation with 4% formaldehyde after 8 hours of infection (single cycle assay).

Like the previous antiviral assay, 2,000 Vero E6 cells were seeded into 96-well plates in DMEM (10% FBS) and incubated for 24 h at 37° C., 5% CO₂. Two hours before infection, the medium was replaced with 100 μL of DMEM (2% FBS) including the combination of HCV protease inhibitors and remdesivir following a dilution combination matrix. A 6 by 6 matrix of drug combinations was prepared in triplicate by making serial two-fold dilutions of the drugs on each axis, including a DMSO control column and row. The resulting matrix had no drug in the right upper well, a single drug in rising 2-fold concentrations in the vertical and horizontal axes starting from that well, and the remaining wells with rising concentrations of drug mixtures reaching maximum concentrations of the drugs at the lower left well. Plates were then transferred into the BSL3 facility and SARS-CoV-2 (MOI 0.025) was added in 50 μL of DMEM (2% FBS). Plates were then incubated for 48 h at 37° C. After infection, cells were fixed with final concentration of 5% formaldehyde for 24 hours prior to being removed from the BSL3 facility. The cells were then immunostained for the viral NP protein using the in-house mAb 1C7 provided by Dr. Thomas Moran, MSSM with a DAPI counterstain. Infected cells (AlexaFluor 488) and total cells (DAPI) were quantified using the Celigo (Nexcelcom) imaging cytometer. Infectivity was measured by the accumulation of viral NP protein in the nucleus of the Vero E6 cells (fluorescence accumulation). Percent infection was quantified as [(Infected cells/Total cells)−Background]*100, and the DMSO control was then set to 100% infection for analysis. The combination antiviral assay was performed in biologically independent triplicates.

The apparent IC₉₀ for each combination in the matrix was determined using the Prism (GraphPad Software) software. The IC₉₀ for HCV drugs and remdesivir were calculated for each drug treatment alone and in combination. This combination data was analyzed using SynergyFinder by the ZIP method, and combination indices were calculated as previously described.

Viral replication measurements were each done in triplicate (n=3), and reported as mean±s.d. Fluorescence measurements were done in triplicate (n=3) or duplicate (n=2) and reported as mean±s.d. Estimates of uncertainties in NMR intensity measurements were determined from the spectral noise, and propagated to uncertainties in peak ratios ΔR as: (ΔR/R)²=(ΔA/A)²+(ΔB/B)², where A, B, and R are the intensities of peaks A and B and their ratio, respectively, and ΔA and ΔB are the noise associated with each intensity measurement.

Methods and systems of the present disclosure are advantageous to provide compositions with combinations of repurposed drugs that target several viral functions and could potentially function as antivirals towards SARS-CoV-2. Several available hepatitis C virus drugs inhibit the SARS-CoV-2 M^{pro} and/or PL^{pro} proteases, and SARS-CoV-2 replication in cell culture. Four HCV drugs that inhibited PL^{pro} enzyme activity also synergize with the viral polymerase inhibitor remdesivir to inhibit virus replication, increasing remdesivir antiviral activity as much as 10-fold. All of these HCV drugs have been approved for use in human clinical trials, and most are FDA-approved prescription drugs, and furthermore can be administered orally. This approach would complement or replace the current standard of care for COVID-19, remdesivir, which is administered by intravenous injection. Such combination therapies should be more effective and have less toxicity and/or side effects than remdesivir alone.

Drug cocktails combining HCV NS3/4A inhibitors together with molecules targeting other SARS-CoV-2 functions, e.g., the viral RdRp, may be valuable as COVID-19 therapeutics. Additionally, repurposed drugs like the HCV drugs described above may not have sufficient inhibitory activity on their own to achieve clinical efficacy. However, synergy with polymerase inhibitors such as remdesivir may increase the potency of both the proposed repurposed HCV drugs and remdesivir, enabling the identification of additional available drugs that can be repurposed as SARS-CoV-2 antivirals and/or as lead molecules for new drug development.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A composition for treating a patient infected with a coronavirus, comprising:
one or more inhibitors of SARS-CoV-2 RNA-dependent RNA polymerase (RdRp), and
one or more inhibitors of SARS-CoV-2 papain-like protease (PL^{pro}) and that are synergistic with the one or more inhibitors for inhibiting SARS-CoV-2 RdRp.

2. The composition according to claim 1, wherein the SARS-CoV-2 RdRp inhibitors include remdesivir, favipiravir, molnupiravir (MK-4482/EIDD-2801), functional equivalents thereof, or combinations thereof.

3. The composition according to claim 1, wherein the SARS-CoV-2 PL^{pro} inhibitors also inhibit hepatitis C virus NS3/4A protease.

4. The composition according to claim 1, wherein the SARS-CoV-2 PL^{pro} inhibitors include grazoprevir, vaniprevir, paritaprevir, GRL0617, Compound 6, or combinations thereof.

5. The composition according to claim 1, wherein the composition includes one or more inhibitors of viral proteins, their complexes, or combinations thereof, that are generated by SARS-CoV-2 PL^{pro} cleavage.

6. The composition according to claim 1, wherein the composition includes inhibitors of non-structural proteins NSP4, NSP6, NSP7, NSP8, NSP9, NSP10, NSP11, NSP12 (RNA polymerase), NSP13, NSP14, NSP15, NSP16, complexes formed between these non-structural proteins, or combinations thereof.

7. The composition according to claim 1, wherein the composition includes:
one or more additional active ingredients; or
one or more pharmaceutically acceptable adjuvants, diluents, excipients, carriers, or combinations thereof.

8. The composition according to claim 1, wherein the composition includes about 5 to about 10-fold less SARS-CoV-2 RdRp inhibitor than the recommended dosage of the SARS-CoV-2 RdRp inhibitor.

9. The composition according to claim 1, wherein the composition includes about 5 to about 10-fold less SARS-CoV-2 PL^{pro} inhibitor than the recommended dosage of the SARS-CoV-2 PL^{pro} inhibitor.

10. A method of treating a patient infected with SARS-CoV-2, comprising:
identifying a SARS-CoV-2 infection in the patient; and
administering an effective amount of a composition to the patient, the composition including:
one or more inhibitors of SARS-CoV-2 RNA-dependent RNA polymerase (RdRp), and
one or more inhibitors of SARS-CoV-2 papain-like protease (PL^{pro}) and that are synergistic with the one or more inhibitors for inhibiting SARS-CoV-2 RdRp.

11. The method according to claim 10, wherein the SARS-CoV-2 RdRp inhibitors include remdesivir, favipiravir, molnupiravir (MK-4482/EIDD-2801), functional equivalents thereof, or combinations thereof.

12. The method according to claim 10, wherein the SARS-CoV-2 PL^{pro} inhibitors also inhibit hepatitis C virus NS3/4A protease.

13. The method according to claim 10, wherein the SARS-CoV-2 PL^{pro} inhibitors include grazoprevir, vaniprevir, paritaprevir, GRL0617, Compound 6, or combinations thereof.

14. The method according to claim 10, wherein the composition includes one or more inhibitors of viral proteins, their complexes, or combinations thereof, that are generated by SARS-CoV-2 PL^{pro} cleavage.

15. The method according to claim 10, wherein the composition includes inhibitors of non-structural proteins NSP4, NSP6, NSP7, NSP8, NSP9, NSP10, NSP11, NSP12 (RNA polymerase), NSP13, NSP14, NSP15, NSP16, complexes formed between these non-structural proteins, or combinations thereof.

16. The method according to claim 10, wherein the composition includes:
one or more additional active ingredients; or
one or more pharmaceutically acceptable adjuvants, diluents, excipients, carriers, or combinations thereof.

17. The method according to claim 10, wherein administering an effective amount of a composition to the patient further comprises:
administering to the patient about 5 to about 10-fold less SARS-CoV-2 RdRp inhibitor than the recommended dosage of the SARS-CoV-2 RdRp inhibitor.

18. The method according to claim 10, wherein administering an effective amount of a composition to the patient further comprises:
administering to the patient about 5 to about 10-fold less SARS-CoV-2 PL^{pro} inhibitor than the recommended dosage of the SARS-CoV-2 PL^{pro} inhibitor.

19. A method of treating a patient infected with SARS-CoV-2, comprising:
identifying a SARS-CoV-2 infection in the patient; and
administering to the patient an effective amount of an inhibitor of SARS-CoV-2 RNA-dependent RNA polymerase (RdRp) and an effective amount of an inhibitor of SARS-CoV-2 papain-like protease (PL^{pro}) that is also synergistic with the SARS-CoV-2 RdRp inhibitor,
wherein the SARS-CoV-2 PL^{pro} inhibitor is paritaprevir,
wherein the effective amount of the SARS-CoV-2 RdRp inhibitor is about 5 to about 10-fold less SARS-CoV-2 RdRp inhibitor than the recommended dosage of the SARS-CoV-2 RdRp inhibitor, and
wherein the effective amount of the SARS-CoV-2 PL^{pro} inhibitor is about 5 to about 10-fold less SARS-CoV-2 PL^{pro} inhibitor than the recommended dosage of the SARS-CoV-2 PL^{pro} inhibitor.

20. The method according to claim 19, wherein the SARS-CoV-2 polymerase inhibitor is orally ingestible by the patient.
